(19) United States
(12) Patent Application Publication
(10) Pub. No.: US 2026/0245355 A1
(43) Pub. Date: Aug. 20, 2026

(54) INLINE AI-BASED MEDICAL IMAGE QUALITY CONTROL SYSTEM

(71) Applicant: Siemens Healthineers AG, Forchheim (DE)

(72) Inventors: Teodora Marina Chitiboi, Hamburg (DE); Puneet Sharma, Princeton Junction, NJ (US)

(21) Appl. No.: 19/056,978

(22) Filed: Feb. 19, 2025

(51) Int. Cl. G06V 10/98; G06V 10/82; G16H 30/40

(52) U.S. Cl. CPC G06V 10/993; G06V 10/82; G16H 30/40

(57) ABSTRACT

Systems and methods for detecting and mitigating imaging errors in medical images. One or more input medical images of an anatomical object of a patient acquired according to an imaging protocol are received. One or more imaging errors are detected in the one or more input medical images using one or more machine learning based networks. The imaging protocol is modified based on the one or more detected imaging errors. The modified imaging protocol is output.

### TECHNICAL FIELD

The present invention relates generally to AI/ML (artificial intelligence/machine learning) based medical imaging analysis, and in particular to an inline AI-based medical image quality control system.

### BACKGROUND

CMR (cardiovascular magnetic resonance) is a non-invasive imaging technique that uses radio waves and magnetic fields to create detailed images of the heart and blood vessels of patients. CMR imaging is important in the diagnosis and management of cardiovascular diseases, enabling the imaging of different physiological aspects of the heart. However, CMR imaging is often lengthy and complicated, involving complex imaging protocols and immediate quality control of the acquired images. Recently, AI-based techniques have been proposed for the quality control of CMR images. However, such conventional AI-based quality control techniques are currently applied independently and retrospectively of the CMR imaging process and are not seamlessly integrated in the image acquisition pipeline, complicating the CMR imaging process.

### BRIEF SUMMARY OF THE INVENTION

In accordance with one or more embodiments, systems and methods for detecting and mitigating imaging errors in medical images. One or more input medical images of an anatomical object of a patient acquired according to an imaging protocol are received. One or more imaging errors are detected in the one or more input medical images using one or more machine learning based networks. The imaging protocol is modified based on the one or more detected imaging errors. The modified imaging protocol is output.

In one embodiment, one or more imaging artifacts are detected in the one or more input medical images using one or more machine learning based imaging artifact detection networks. The one or more imaging artifacts comprise at least one of image-based artifacts or k-space-based artifacts.

In one embodiment, the one or more machine learning based imaging artifact detection networks are trained using synthetic images with imaging artifacts generated using a generator network. The generator network receives as input real images and image artifact settings and generates as output the synthetic images with the imaging artifacts.

In one embodiment, the one or more machine learning based imaging artifact detection networks are trained using synthetic images with imaging artifacts generated using a first generator network and a second generator network. The first generator network receives as input real k-space data and generates as output synthetic k-space data. The synthetic k-space data is reconstructed into the synthetic images with the imaging artifacts. The second generator network receives as input real images and generates as output the synthetic images with the imaging artifacts.

In one embodiment, the first generator network and the second generator network are trained to enforce consistency between synthetic k-space training data generated by the first generator network and synthetic training images generated by the second generator network.

In one embodiment, the one or more input medical images comprise one or more raw medical images and a plurality of post-processed medical images. One or more post-processing errors are detected in one or more of the plurality of post-processed medical images using a machine learning based post-processing error detection network. The machine learning based post-processing error detection network receives as input the one or more raw medical images and the plurality of post-processed medical images and generates as output a plausibility classification for each of the plurality of post-processed medical images.

In one embodiment, one or more imaging artifacts are detected in the one or more input medical images. The one or more detected imaging artifacts are propagated to a post-processing error using a mapping function.

In one embodiment, the one or more input medical images are automatically reacquired or reprocessed according to the modified imaging protocol to acquire one or more additional medical images having the one or more detected imaging artifacts mitigated.

These and other advantages of the invention will be apparent to those of ordinary skill in the art by reference to the following detailed description and the accompanying drawings.

### BRIEF DESCRIPTION OF THE DRAWINGS

FIG. 1 shows a method for detecting and mitigating imaging errors in one or more input medical images, in accordance with one or more embodiments;

FIG. 2 shows a workflow for training a GAN (generative adversarial network) for generating synthetic images with imaging artifacts in a second modality based on images of a first modality, in accordance with one or more embodiments;

FIG. 3 shows a workflow for training a GAN for generating synthetic images with imaging artifacts based on images and k-space data, in accordance with one or more embodiments;

FIG. 4 shows an exemplary artificial neural network that may be used to implement one or more embodiments;

FIG. 5 shows a convolutional neural network that may be used to implement one or more embodiments;

FIG. 6 shows a data flow diagram according to an embodiment for using a GAN, in accordance with one or more embodiments;

FIG. 7 shows a schematic structure of a recurrent machine learning model that may be used to implement one or more embodiments; and

FIG. 8 shows a high-level block diagram of a computer that may be used to implement one or more embodiments.

### DETAILED DESCRIPTION

The present invention generally relates to methods and systems for inline AI-based medical image quality control. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system. Further, reference herein to pixels of an image may refer equally to voxels of an image and vice versa.

Embodiments described herein provide for an AI-based medical image quality control system implemented inline on an image acquisition device. The image quality control system detects imaging errors in input medical images using one or more machine learning based networks and modifies the imaging protocol to mitigate the detected imaging errors for reacquiring or re-post-processing. Such imaging errors may comprise, for example, image artifacts or post-processing errors. The one or more machine learning based networks are trained to detect the imaging errors using synthetic training data generated to depict such imaging errors. Advantageously, embodiments described herein provide for an improved image quality control system that operates inline on an image acquisition device at the time of image acquisition to provide immediate interactive feedback to users, thereby reducing image acquisition time and complexity while providing for improved image quality as compared with conventional approaches.

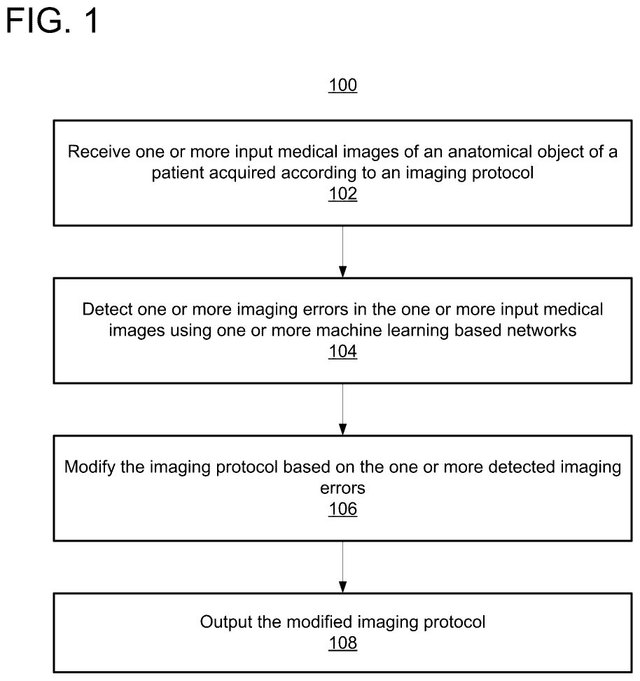

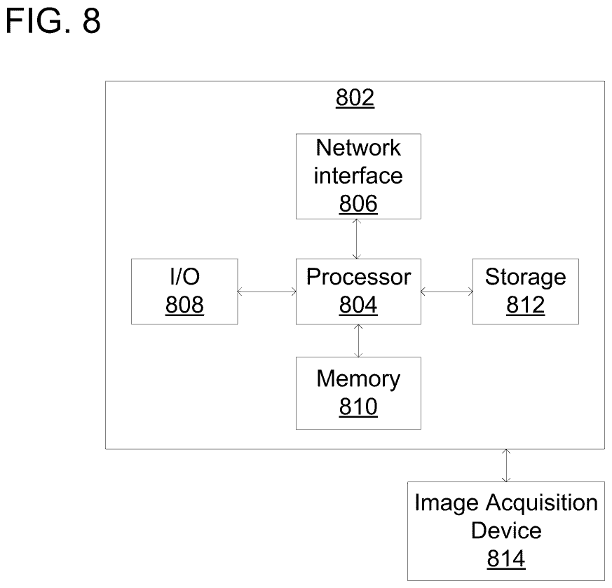

FIG. 1 shows a method 100 for detecting and mitigating imaging errors in one or more input medical images, in accordance with one or more embodiments. The steps and sub-steps of method 100 may be performed by one or more suitable computing devices, such as, e.g., computer 802 of FIG. 8.

At step 102 of FIG. 1, one or more input medical images of an anatomical object of a patient acquired according to an imaging protocol is received. The anatomical object may comprise, for example, organs, bones, vessels, lesions or other abnormalities, or any other suitable anatomical object of the patient. In one embodiment, the one or more input medical images are received upon acquisition by an image acquisition device (e.g., image acquisition device 814 of FIG. 8).

In one embodiment, the one or more input medical images comprise MRI (magnetic resonance imaging) images of a chest (e.g., heart and vessels) of the patient. However, the one or more input medical images may comprise images of any other suitable modality, such as, CT (computed tomography), US (ultrasound), x-ray, or any other medical imaging modality or combinations of medical imaging modalities. The one or more input medical images may comprise 2D (two dimensional) images and/or a 3D (three dimensional) volumes, and may comprise a single input medical image or a plurality of input medical images. The one or more input medical images may comprise raw images received from the image acquisition device and/or post-processed images resulting from post-processing of the raw images (e.g., for segmentation or registration).

The one or more input medical images are acquired according to the imaging protocol. The imaging protocol comprises a set of parameters that define how the one or more input medical images are acquired. For example, the imaging protocol may define parameters for patient preparation (e.g., pharmacological stress, contrast administration, positioning), machine settings (e.g., radiation dose, exposure time, field of view, slice thickness, acquisition angles), imaging sequences (e.g., T1/T2 weighted MMIR or contrast-enhanced CT), imaging sequence parameters (e.g., repetition time, echo time, flip angle, number of averages, etc.), and post-processing (e.g., image reconstruction techniques, registration or segmentation algorithm settings).

The one or more input medical images may be received, for example, by directly receiving the input medical images from the image acquisition device (e.g., image acquisition device 814 of FIG. 8) as the input medical images are acquired, by loading the input medical images from a storage or memory of a computer system (e.g., storage 812 or memory 810 of computer 802 of FIG. 8), or by receiving the input medical images from a remote computer system (e.g., computer 802 of FIG. 8). Such a computer system or remote computer system may comprise one or more patient databases, such as, e.g., an EHR (electronic health record), EMR (electronic medical record), PHR (personal health record), HIS (health information system), RIS (radiology information system), PACS (picture archiving and communication system), LIMS (laboratory information management system), or any other suitable database or system.

At step 104 of FIG. 1, one or more imaging errors are detected in the one or more input medical images using one or more machine learning based networks. The one or more imaging errors may comprise, for example, imaging artifacts, post-processing errors, or any other imaging error.

In one embodiment, the one or more imaging errors comprise imaging artifacts. Imaging artifacts are unwanted distortions, abnormalities, or errors in the one or more input medical images. The imaging artifacts may be caused by, for example, patient movement, hardware limitations, scanning techniques, or process errors. The imaging artifacts may comprise, for example, image-based artifacts, k-space-based artifacts, or artifacts that are in both the image space and the k-space (e.g., a combination of image-based artifacts and k-space-based artifacts).

Image-based artifacts are artifacts that occur during the processing, reconstruction, or acquisition of the one or more input medical images. Exemplary image-based artifacts include incorrect field of view, acquisition volume or slice location, image degradation caused by patient or object motion, for example due to breathing or heartbeat, susceptibility artifacts caused by implanted metal objects in the patient, truncation artifacts (Gibbs or ringing artifact), or chemical shift.

K-space-based artifacts are artifacts that occur during the collection, organization, or processing of k-space data for the one or more input medical images. K-space is a mathematical data storage matrix used in MRI to represent raw frequency and phase information before image reconstruction into the one or more input medical images. Exemplary k-space-based artifacts include motion artifacts (due to patient movement altering phase encoding, resulting in misalignment in k-space), aliasing (due to under sampling in k-space when the field of view is too small), truncation (due to insufficient k-space sampling), zipper artifacts (due to external radiofrequency interference contaminating k-space), spike noise artifacts (sudden spikes or missing data points in k-space due to radiofrequency noise or scanner malfunctions), parallel imaging artifacts (errors in k-space reconstruction when using parallel imaging), or k-space filling artifacts (incomplete or irregular sampling of k-space).

The imaging artifacts may be detected using one or more machine learning based imaging artifact detection networks. The one or more machine learning based imaging artifact detection networks detect a presence of different types of imaging artifacts in the one or more input medical images. The one or more machine learning based imaging artifact detection networks receive as input the one or more input medical images and generate as output a score (e.g., from 0 to 1) for each type of imaging artifacts. The score for a particular imaging artifact represents a probability that the particular imaging artifact is present in the one or more input medical images.

In one embodiment, the one or more machine learning based imaging artifact detection networks comprise an ensemble of machine learning based imaging artifact detection networks, each trained to detect and generate a score for a subset of imaging artifacts. For example, the ensemble may comprise a first machine learning based imaging artifact detection network for detecting image-based artifacts, a second machine learning based imaging artifact detection network for detecting k-space-based artifacts, and third machine learning based imaging artifact detection network for detecting both image-based artifacts and k-space-based artifacts.

The one or more machine learning based imaging artifact detection networks are trained during a prior offline or training stage. Once trained, the one or more machine learning based imaging artifact detection networks are applied during an online or inference stage, e.g., to perform step 104 of FIG. 1.

In one embodiment, the one or more machine learning based imaging artifact detection networks are trained using synthetic images with imaging artifacts. For example, synthetic images with image-based artifacts may be generated by: 1) generating synthetic images having an incorrect field of view where the anatomical object is not fully covered, 2) generating synthetic images having an incorrect orientation by simulation a 2D incorrect orientation acquisition using a 3D volume acquisition as a starting point, 3) generating synthetic images having breathing motion artifacts by artificially shifting different images in a temporal series acquisition and recombing the results, 4) generating synthetic images having heartbeat motion by simulating triggering/arrhythmia motion artifacts by recombining images acquired in different phases from an existing image series, 5) generating synthetic images having susceptibility artifacts by simulating the implantation of metal device on existing images, 6) generating synthetic images having truncation artifacts (that appear as concentric rings or lines near sharp borders), and/or 7) generating synthetic images having chemical shift artifacts. In another example, synthetic images with k-space-based artifacts may be generated by: 1) under sampling, 2) aliasing, 3) different levels of noise, and/or 4) motion artifacts.

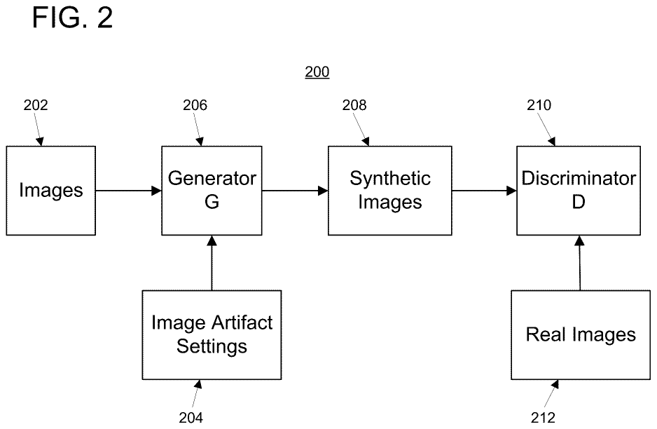

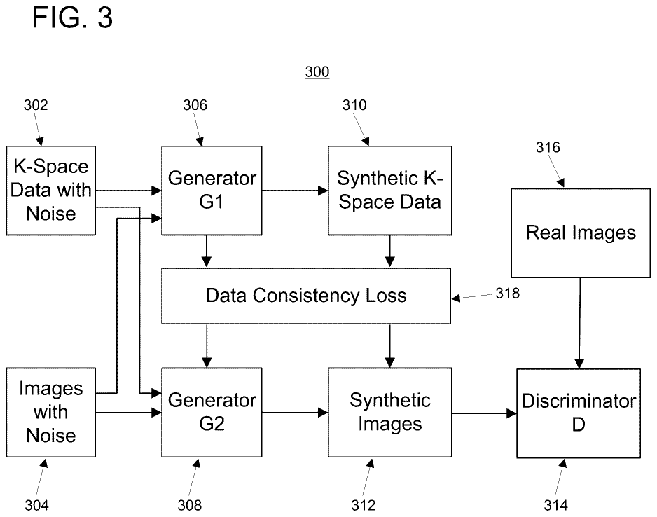

In one embodiment, the synthetic images with imaging artifacts may be generated using a GAN (generative adversarial network) without the need for numerical simulation. The GAN may be trained using unpaired data with and without different types of artifacts. In one embodiment, the GAN may generate synthetic images with imaging artifacts in a second modality based on images of a first modality. The GAN in this embodiment may be trained according to FIG. 2, discussed in detail below. In another embodiment, the GAN may generate synthetic images with imaging artifacts based on k-space data and image data. The GAN in this embodiment may be trained according to FIG. 3, discussed in detail below.

FIG. 2 shows a workflow 200 for training a GAN for generating synthetic images with imaging artifacts in a second modality based on images of a first modality, in accordance with one or more embodiments. In workflow 200, generator G 206 receives images 202 of a first modality as well as image artifact settings 204 and generates as output synthetic images 208. Generator G 206 may be implemented as a deep neural network, such as, e.g., a DI2IN (deep image-to-image network) implemented using a convolutional encoder-decoder architecture, but may be implemented according to any other suitable machine learning based architecture. In one embodiment, images 202 comprise 3D CT volumes and synthetic images 208 comprise 2D MRI slices with imaging artifacts. However, images 202 and synthetic images 208 may comprise images of any other modality or dimension.

Image artifact settings 204 define the desired imaging artifact to be generated in synthetic images 208. For example, synthetic images 208 may comprise images having an incorrect field of view with part of the anatomical object missing or images having an incorrect orientation (e.g., a long axis acquisition that does not depict the atrium/atria or does not pass through the heart apex). In this example, image artifact settings 204 may comprise a 3D plane through images 202 that produces a 2D imaging plane with the wrong field of view or orientation. Discriminator D 210 compares the synthetic images 208 with real images 212 (with or without imaging artifacts). Discriminator D 210 may also be implemented as a deep neural network that distinguishes between the synthetic images 208 and the real images 212.

During training, generator G 206 and discriminator D 210 are updated dynamically in the sense of learning better network parameters until they reach equilibrium, that is, the synthetic images 208 becomes as close as possible from being indistinguishable from the real images 212 through the eyes of the discriminator D 210. The trained generator G 206 is then stored, for example, in a memory or storage of a computer system and used alone (i.e., without discriminator D 210) for inference. For example, during inference, the trained generator G 206 receives as input real images and image artifact settings and generates as output synthetic images with imaging artifacts for training the one or more machine learning based imaging artifact detection networks.

FIG. 3 shows a workflow 300 for training a GAN for generating synthetic images with imaging artifacts based on images and k-space data, in accordance with one or more embodiments. In workflow 300, generator G1 306 and generator G2 308 both receive as input k-space data with noise 302 and images with noise 304. Generator G1 306 and generator G2 308 may be implemented as deep neural networks, but may be implemented according to any other suitable machine learning based architecture. The noise of k-space data with noise 302 may comprise the omission or some k-space points (under sampling) while the noise of images with noise 304 may be numerically simulated imaging artifacts (e.g., susceptibility artifacts). The noise in k-space data with noise 302 or images with noise 304 may or may not be random. The noise enables the same real k-space data or images to be used multiple times as input with different noise maps added to them. Generator G1 306 generates synthetic k-space data 310 and generator G2 308 generates synthetic images 312. In one embodiment, images with noise 304 and synthetic images 312 are 3D MRI images, but may be of any other modality or dimension. Discriminator D 314 compares the synthetic images 312 with real images 316 (with or without imaging artifacts). Discriminator D 314 may also be implemented as a deep neural network that distinguishes between the synthetic images 312 and the real images 316.

During training, generators G1 306 and G2 308 and discriminator D 314 are updated dynamically in the sense of learning better network parameters until they reach equilibrium, that is, the synthetic images 312 becomes as close as possible from being indistinguishable from the real images 316 through the eyes of the discriminator D 314. Generators G1 306 and G2 308 are also trained with data consistency loss 318 to enforce consistence between synthetic k-space data 310 and synthetic images 312. The trained generators G1 306 and G2 308 are then stored, for example, in a memory or storage of a computer system and used alone (i.e., without discriminator D 314) for inference. For example, during inference, generator G1 306 may receive as input real k-space data and generates as output synthetic k-space data, which may be reconstructed into synthetic images with imaging artifacts for training the one or more machine learning based imaging artifact detection networks. In another example, during inference, generator G2 308 may receive as input images and generates as output synthetic images with imaging artifacts for training the one or more machine learning based imaging artifact detection networks.

Returning back to step 104 of FIG. 1, the one or more machine learning based imaging artifact detection networks are trained using synthetic images with imaging artifacts. The one or more machine learning based imaging artifact detection networks may be fine-tuned on real data with and without imaging artifacts. In some embodiments, the one or more machine learning based imaging artifact detection networks may also be pretrained on unlabeled data where the presence of artifacts is uncertain. The one or more machine learning based imaging artifact detection networks may be applied during inference inline on the image acquisition device at the time of acquisition of the one or more input medical images to inform the clinician of acquisition problems and trigger reacquisition or may be applied offline as a pure post-processing step to exclude images with artifacts from further post-processing or analysis.

In one embodiment, the one or more imaging errors comprise post-processing errors. Post-processing errors are errors that occur during the image reconstruction, enhancement, or analysis after the raw imaging data has been acquired. Post-processing errors may include, for example, misregistration errors or segmentation errors. The post-processing errors may be detected using a machine learning based post-processing error detection network. The machine learning based post-processing error detection network receives as input the one or more input medical images (comprising a raw image received from the image acquisition device and one or more post-processed images) and outputs a plausibility classification of the one or more post-processed images as being plausible or not plausible (i.e., implausible). The plausibility classification may be represented as a continuous value (e.g., between 0 and 1 representing a probability of plausibility), a discrete value (e.g., either 0 or 1 representing a binary plausible or not plausible classification), or in any other suitable form.

The machine learning based post-processing error detection network is trained during a prior offline or training stage. Once trained, the machine learning based post-processing error detection network may be applied during an online or inference stage, e.g., to perform step 104 of FIG. 1. In one embodiment, the machine learning based post-processing error detection network may be trained using synthetic data simulating different types of post-processing errors. For example, segmentation errors may be simulated by altering the segmentation mask or misregistration errors may be simulated by introducing misalignment into a set of aligned data.

In one embodiment, a rule-based algorithm can be used to detect the post-processing errors. In another embodiment, the machine learning based post-processing error detection network may be used in combination with the rule-based algorithm. For example, a rule-based algorithm may be used in combination with the machine learning based post-processing error detection network to analyze the quality of co-registration results based on the consistency of the segmentation mask for different co-registered images. In this example, to check the consistency of the segmentation mask, either a rule-based decision may be applied (e.g., all masks should have a 90% overlap) or a machine learning based post-processing error detection network may be trained with artificially generated masks that either have a good temporal consistency or were artificially distorted to have a poorer overlay. When combining both these tests, one could flag a post-processing error when either one of the tests fail or when both tests fail. This is particularly beneficial when trying to predict the plausibility of, for example, an automatically segmented organ mask. Where one could have a statistical model of the organ and flag the cases that fit this model poorly (e.g., a direct mathematical test with a threshold) or train a neural network to give a confidence score to each pixel of the masks. This network may be trained using artificially generated wrong segmentations starting from correct segmentation by introducing small deformation. Having these two independent tests of the mask plausibility, one could apply both of them either using “or” or “and” to combine the results. In another example, a rule-based algorithm may be used in combination with the machine learning based post-processing error detection network to analyze the quality of segmentation result based on the consistency of volume and mass of different structures, along the temporal series. For example, the stroke volume of the left and right ventricles should be similar in value, except for rare diseased cases.

In one embodiment, post-processing errors may be predicted based on the imaging artifacts. Different imaging artifacts can decrease the quality of the resulting image and directly affect the performance of post-processing by introducing errors in the post-processing results. Error propagation may be performed to predict post-processing errors based on imaging artifacts. For the machine learning based post-processing error detection network, the error propagation of different types of artifacts may be quantified through a pipeline similar to a Monte-Carlo simulation. Different synthetic images may be created with given imaging artifacts of different levels of severity, such as, e.g., 2D images with a different misalignment angle form the optimal acquisition position or 2D image stacks which miss a different part of the target anatomy, or susceptibility artifacts at a different position (e.g., closer or further from the target anatomy). Through repeated simulation, the effect of the synthetic artifacts may be quantified on the results of the post-processing. Based on the simulation, a mapping function FER can be learned (by different types of machine learning algorithms, such as, e.g., regression or a neural network) between the imaging artifact type and intensity and the resulting error introduced. To implement error propagation estimation on an image acquisition device, mapping function FER can be applied to newly acquired medical images to predict what error the detected imaging artifact may produce in the post-processing result.

At step 106 of FIG. 1, the imaging protocol is modified based on the one or more detected imaging errors. In one embodiment, the imaging protocol may be modified to mitigate the one or more detected imaging errors based on exiting knowledge. For example, the modification to the imaging protocol may be to increase the field of view, reduce the TR (repetition time), TE (echo time), etc., re-adjust the shim box, etc. In another example, the modification to the imaging protocol may be a suggested placement of a saturation band for that type of anatomy. In one embodiment, the available hardware and software that the clinician is using is analyzed to identify unusual activity.

In one embodiment, the modification to the imaging protocol is rule-based, e.g., based on the current protocol configuration and the type of artifact detected. In another embodiment, a language model (e.g., an LLM, large language model) is adapted to modify the imaging protocol to improve image quality. The language model would receive as input a prompt with the most relevant sequence parameters used and the type of artifact detected and provide different modifications to the imaging protocol. This intermediate prompting step would be hidden to the end user. The prompting may be triggered automatically in the background if an artifact is detected.

At step 108 of FIG. 1, the modified imaging protocol is output. For example, the modified imaging protocol can be output by displaying the results on a display device of a computer system (e.g., I/O 808 of computer 802 of FIG. 8), storing the modified imaging protocol on a memory or storage of a computer system (e.g., memory 810 or storage 812 of computer 802 of FIG. 8), or by transmitting the modified imaging protocol to a remote computer system (e.g., computer 802 of FIG. 8).

In one embodiment, the modified imaging protocol may be presented to the clinician. The clinician may be prompted by the one or more machine learning based networks immediately upon image acquisition regarding the presence of the one or more detected imaging errors based on the score the machine learning based network predicts for each artifact type. For example, the modified imaging protocol may be presented to the clinician by directly navigating to the specific sub-menu in the image acquisition device, highlighting the parameter that should be modified, and presenting the clinician with the suggested modification. The clinician can then determine whether to reacquire or re-post-process the one or more input medical images according to the modified imaging protocol to acquire one or more additional medical images. The one or more additional medical images have the one or more detected errors mitigated.

In one embodiment, the one or more input medical images are automatically reacquired or reprocessed (to reperform the post-processing) according to the modified imaging protocol to acquire one or more additional medical images. In another embodiment, the one or more input medical images are automatically reacquired or reprocessed according to the top N modified imaging protocols to acquire one or more additional medical images (where N is any positive integer). The one or more additional medical images for the top N modified imaging protocols may be output (e.g., presented) to the clinician. For example, additional images may be acquired with an increased field of view, shorter TR, and a refitted shim box. The three images may be presented to the clinician and the clinician may select the best image. In another example, post-processing can be retriggered with different parameters that are most likely to influence the result, such as, e.g., retriggering a segmentation step with different positions of key landmarks (e.g., valve landmarks or ventricle base) or retriggering an image registration with different levels or regularization. The clinician's selection may be pre-configured for different types of workflows to favor one type of solution over other. The clinician's choice may be collected over time to improve the algorithm.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, a machine learning model, such as, e.g., the one or more machine learning based networks utilized at step 104 of FIG. 1, generator G 206 and discriminator D 210 of FIG. 2, and generators G1 306 and G2 308 and discriminator D 314 of FIG. 3, can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

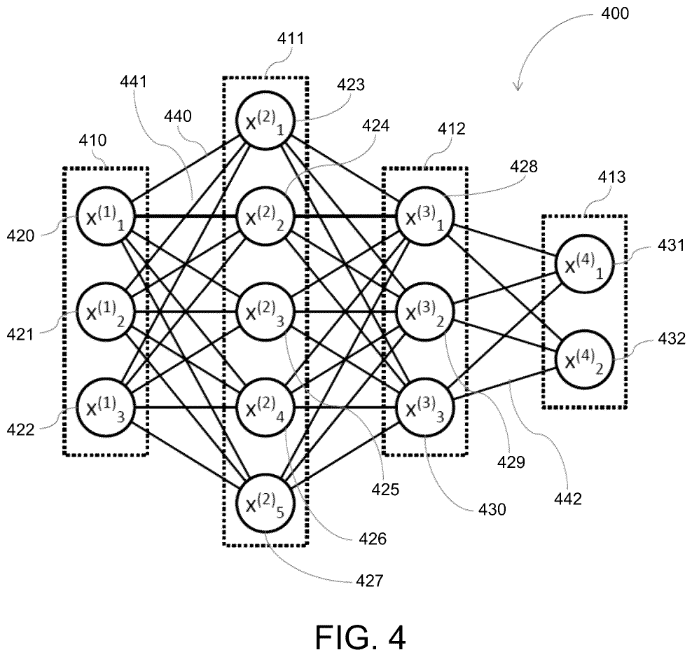

FIG. 4 shows an embodiment of an artificial neural network 400 that may be used to implement one or more machine learning models described herein. Alternative terms for “artificial neural network” are “neural network”, “artificial neural net” or “neural net”.

The artificial neural network 400 comprises nodes 420, . . . , 432 and edges 440, . . . , 442, wherein each edge 440, . . . , 442 is a directed connection from a first node 420, . . . , 432 to a second node 420, . . . , 432. In general, the first node 420, . . . , 432 and the second node 420, . . . 432 are different nodes 420, . . . , 432, it is also possible that the first node 420, . . . , 432 and the second node 420, . . . , 432 are identical. For example, in FIG. 4 the edge 440 is a directed connection from the node 420 to the node 423, and the edge 442 is a directed connection from the node 430 to the node 432. An edge 440, . . . , 442 from a first node 420, . . . , 432 to a second node 420, . . . , 432 is also denoted as “ingoing edge” for the second node 420, . . . , 432 and as “outgoing edge” for the first node 420, . . . , 432.

In this embodiment, the nodes 420, . . . , 432 of the artificial neural network 400 can be arranged in layers 410, . . . , 413, wherein the layers can comprise an intrinsic order introduced by the edges 440, . . . , 442 between the nodes 420, . . . , 432. In particular, edges 440, . . . , 442 can exist only between neighboring layers of nodes. In the displayed embodiment, there is an input layer 410 comprising only nodes 420, . . . , 422 without an incoming edge, an output layer 413 comprising only nodes 431, 432 without outgoing edges, and hidden layers 411, 412 in-between the input layer 410 and the output layer 413. In general, the number of hidden layers 411, 412 can be chosen arbitrarily. The number of nodes 420, . . . , 422 within the input layer 410 usually relates to the number of input values of the neural network, and the number of nodes 431, 432 within the output layer 413 usually relates to the number of output values of the neural network.

In particular, a (real) number can be assigned as a value to every node 420, . . . , 432 of the neural network 400. Here, x(n); denotes the value of the i-th node 420, . . . , 432 of the n-th layer 410, . . . , 413. The values of the nodes 420, . . . , 422 of the input layer 410 are equivalent to the input values of the neural network 400, the values of the nodes 431, 432 of the output layer 413 are equivalent to the output value of the neural network 400. Furthermore, each edge 440, . . . , 442 can comprise a weight being a real number, in particular, the weight is a real number within the interval [−1, 1] or within the interval [0, 1]. Here, w^{(m,n)}_{i,j} denotes the weight of the edge between the i-th node 420, . . . , 432 of the m-th layer 410, . . . , 413 and the j-th node 420, . . . , 432 of the n-th layer 410, . . . , 413. Furthermore, the abbreviation w⁽ⁿ⁾_{i,j} is defined for the weight w^{(n,n+1)}_{i,j}.

In particular, to calculate the output values of the neural network 400, the input values are propagated through the neural network. In particular, the values of the nodes 420, . . . , 432 of the (n+1)-th layer 410, . . . , 413 can be calculated based on the values of the nodes 420, . . . 432 of the n-th layer 410, . . . , 413 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 410 are given by the input of the neural network 400, wherein values of the first hid-den layer 411 can be calculated based on the values of the input layer 410 of the neural network, wherein values of the second hidden layer 412 can be calculated based in the values of the first hidden layer 411, etc.

In order to set the values w^{(m,n)}_{i,j} for the edges, the neural network 400 has to be trained using training data. In particular, training data comprises training input data and training output data (denoted as tᵢ). For a training step, the neural network 400 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 400 (backpropagation algorithm). In particular, the weights are changed according to

$${w}^{{′ ( n )}_{i , j}} = {w}^{{( n )}_{i , j}} - γ \cdot {δ}^{{( n )}_{j}} \cdot {x}^{{( n )}_{i}}$$

wherein γ is a learning rate, and the numbers δ⁽ⁿ⁾; can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 413, wherein f′ is the first derivative of the activation function, and t⁽ⁿ⁺¹⁾; is the comparison training value for the j-th node of the output layer 413.

A convolutional neural network is a neural network that uses a convolution operation instead of general matrix multiplication in at least one of its layers (so-called “convolutional layer”). In particular, a convolutional layer performs a dot product of one or more convolution kernels with the convolutional layer's input data/image, wherein the entries of the one or more convolution kernels are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

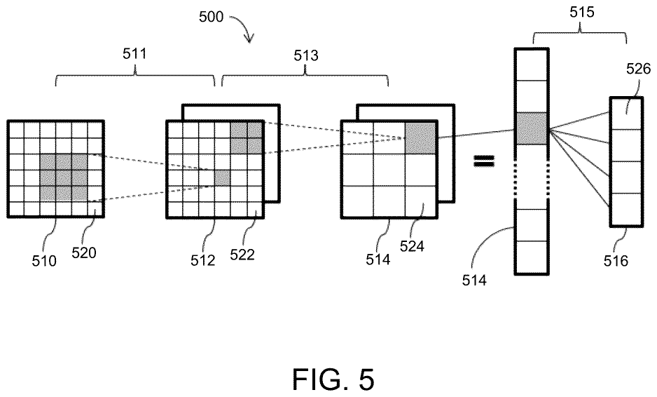

FIG. 5 shows an embodiment of a convolutional neural network 500 that may be used to implement one or more machine learning models described herein. In the displayed embodiment, the convolutional neural network 500 comprises an input node layer 510, a convolutional layer 511, a pooling layer 513, a fully connected layer 514 and an output node layer 516, as well as hidden node layers 512, 514. Alternatively, the convolutional neural network 500 can comprise several convolutional layers 511, several pooling layers 513 and several fully connected layers 515, as well as other types of layers. The order of the layers can be chosen arbitrarily, usually fully connected layers 515 are used as the last layers before the output layer 516.

In particular, within a convolutional neural network 500 nodes 520, 522, 524 of a node layer 510, 512, 514 can be considered to be arranged as a d-dimensional matrix or as a d-dimensional image. In particular, in the two-dimensional case the value of the node 520, 522, 524 indexed with i and j in the n-th node layer 510, 512, 514 can be denoted as x(n)[i, j]. However, the arrangement of the nodes 520, 522, 524 of one node layer 510, 512, 514 does not have an effect on the calculations executed within the convolutional neural network 500 as such, since these are given solely by the structure and the weights of the edges.

A convolutional layer 511 is a connection layer between an anterior node layer 510 (with node values x(n−1)) and a posterior node layer 512 (with node values x(n)). In particular, a convolutional layer 511 is characterized by the structure and the weights of the incoming edges forming a convolution operation based on a certain number of kernels. In particular, the structure and the weights of the edges of the convolutional layer 511 are chosen such that the values x(n) of the nodes 522 of the posterior node layer 512 are calculated as a convolution x(n)=K*x(n−1) based on the values x(n−1) of the nodes 520 anterior node layer 510, where the convolution * is defined in the two-dimensional case as

$${x}_{k}^{( n )} [ i , j ] = ( K * {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

Here the kernel K is a d-dimensional matrix (in this embodiment, a two-dimensional matrix), which is usually small compared to the number of nodes 520, 522 (e.g., a 3×3 matrix, or a 5×5 matrix). In particular, this implies that the weights of the edges in the convolution layer 511 are not independent, but chosen such that they produce said convolution equation. In particular, for a kernel being a 3×3 matrix, there are only 9 independent weights (each entry of the kernel matrix corresponding to one independent weight), irrespectively of the number of nodes 520, 522 in the anterior node layer 510 and the posterior node layer 512.

In general, convolutional neural networks 500 use node layers 510, 512, 514 with a plurality of channels, in particular, due to the use of a plurality of kernels in convolutional layers 511. In those cases, the node layers can be considered as (d+1)-dimensional matrices (the first dimension indexing the channels). The action of a convolutional layer 511 is then a two-dimensional example defined as

$${x}^{{( n )}_{b}} [ i , j ] = {\sum }_{a} {K}_{a , b} * {x}^{{( n - 1 )}_{a}} [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ]$$

where x⁽ⁿ⁻¹⁾ a corresponds to the a-th channel of the anterior node layer 510, x⁽ⁿ⁾_{b} corresponds to the b-th channel of the posterior node layer 512 and K_{a,b} corresponds to one of the kernels. If a convolutional layer 511 acts on an anterior node layer 510 with A channels and outputs a posterior node layer 512 with B channels, there are A.B independent d-dimensional kernels K_{a,b}.

In general, in convolutional neural networks 500 activation functions are used. In this embodiment ReLU (acronym for “Rectified Linear Units”) is used, with R(z)=max(0, z), so that the action of the convolutional layer 511 in the two-dimensional example is

$${x}^{{( n )}_{b}} [ i , j ] = R ( {\sum }_{a} ( {K}_{a , b} * {x}^{{( n - 1 )}_{a}} ) [ i , j ] ) = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tanh or Softmax.

In the displayed embodiment, the input layer 510 comprises 36 nodes 520, arranged as a two-dimensional 6×6 matrix. The first hidden node layer 512 comprises 72 nodes 522, arranged as two two-dimensional 6×6 matrices, each of the two matrices being the result of a convolution of the values of the input layer with a 3×3 kernel within the convolutional layer 511. Equivalently, the nodes 522 of the first hidden node layer 512 can be interpreted as arranged as a three-dimensional 2×6×6 matrix, wherein the first dimension correspond to the channel dimension.

The advantage of using convolutional layers 511 is that spatially local correlation of the input data can exploited by enforcing a local connectivity pattern between nodes of adjacent layers, in particular by each node being connected to only a small region of the nodes of the preceding layer.

A pooling layer 513 is a connection layer between an anterior node layer 512 (with node values x(n−1)) and a posterior node layer 514 (with node values x(n)). In particular, a pooling layer 513 can be characterized by the structure and the weights of the edges and the activation function forming a pooling operation based on a non-linear pooling function f. For example, in the two-dimensional case the values x(n) of the nodes 524 of the posterior node layer 514 can be calculated based on the values x(n−1) of the nodes 522 of the anterior node layer 512 as

$${x}^{{( n )}_{b}} [ i , j ] = f ( {x}^{( n - 1 )} [ {id}_{1} , {jd}_{2} ] , … , {x}^{{( n - 1 )}_{b}} [ ( i + 1 ) {d}_{1} - 1 , ( j + 1 ) {d}_{2} - 1 ] )$$

In other words, by using a pooling layer 513 the number of nodes 522, 524 can be reduced, by re-placing a number d1·d2 of neighboring nodes 522 in the anterior node layer 512 with a single node 522 in the posterior node layer 514 being calculated as a function of the values of said number of neighboring nodes. In particular, the pooling function f can be the max-function, the average or the L2-Norm. In particular, for a pooling layer 513 the weights of the incoming edges are fixed and are not modified by training.

The advantage of using a pooling layer 513 is that the number of nodes 522, 524 and the number of parameters is reduced. This leads to the amount of computation in the network being reduced and to a control of overfitting.

In the displayed embodiment, the pooling layer 513 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 500 are fully connected layers 515. A fully connected layer 515 is a connection layer between an anterior node layer 514 and a posterior node layer 516. A fully connected layer 513 can be characterized by the fact that a majority, in particular, all edges between nodes 514 of the anterior node layer 514 and the nodes 516 of the posterior node layer are present, and wherein the weight of each of these edges can be adjusted individually.

In this embodiment, the nodes 524 of the anterior node layer 514 of the fully connected layer 515 are displayed both as two-dimensional matrices, and additionally as non-related nodes (indicated as a line of nodes, wherein the number of nodes was reduced for a better presentability). This operation is also denoted as “flattening”. In this embodiment, the number of nodes 526 in the posterior node layer 516 of the fully connected layer 515 smaller than the number of nodes 524 in the anterior node layer 514. Alternatively, the number of nodes 526 can be equal or larger.

Furthermore, in this embodiment the Softmax activation function is used within the fully connected layer 515. By applying the Softmax function, the sum the values of all nodes 526 of the output layer 516 is 1, and all values of all nodes 526 of the output layer 516 are real numbers between 0 and 1. In particular, if using the convolutional neural network 500 for categorizing input data, the values of the output layer 516 can be interpreted as the probability of the input data falling into one of the different categories.

In particular, convolutional neural networks 500 can be trained based on the backpropagation algorithm. For preventing overfitting, methods of regularization can be used, e.g., dropout of nodes 520, . . . , 524, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

1. Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

A generative adversarial model (an acronym is GA model) comprises a generative function and a discriminative function, wherein the generative function creates synthetic data, and the discriminative function distinguishes between synthetic and real data. By training the generative function and/or the discriminative function on the one hand the generative function is configured to create synthetic data which is incorrectly classified by the discriminative function as real, on the other hand the discriminative function is configured to distinguish between real data and synthetic data generated by the generative function. In the notion of game theory, a generative adversarial model can be interpreted as a zero-sum game. The training of the generative function and/or of the discriminative function is based, in particular, on the minimization of a cost function.

By using a GA model, based on a set of training data synthetic data can be generated that has the same characteristics as the training data set. The training of the GA model can be based on data not being annotated (unsupervised learning), so that there is low effort in training a GA model.

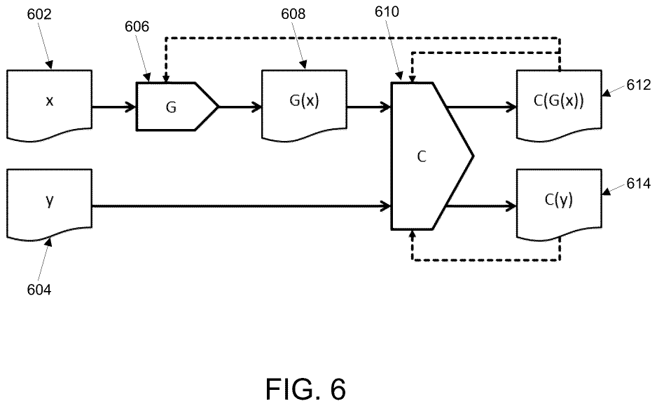

FIG. 6 shows a data flow diagram according to an embodiment for using a generative adversarial network for creating synthetic output data G(x) 608 based on input data x 602 that is indistinguishable from real output data y 604, in accordance with one or more embodiments. The synthetic output data G(x) 608 has the same structure as the real output data y 604, but its content is not derived from real world data.

The generative adversarial network comprises a generator function G 606 and a classifier function C 610 which are trained jointly. The task of the generator function G 606 is to provide realistic synthetic output data G(x) 608 based on input data x 602, and the task of the classifier function C 610 is to distinguish between real output data y 604 and synthetic output data G(x) 608. In particular, the output of the classifier function C 610 is a real number between 0 and 1 corresponding to the probability of the input value being real data, so that an ideal classifier function would calculate an output value of C(y) 614≈1 for real data y 604 and C(G(x)) 612≈0 for synthetic data G(x) 608.

Within the training process, parameters of the generator function G 606 are adapted so that the synthetic output data G(x) 608 has the same characteristics as real output data y 604, so that the classifier function C 610 cannot distinguish between real and synthetic data anymore. At the same time, parameters of the classifier function C 610 are adapted so that it distinguishes between real and synthetic data in the best possible way. Here, the training relies on pairs comprising input data x 602 and the corresponding real output data y 604. Within a single training step, the generator function G 606 is applied to the input data x 602 for generating synthetic output data G(x) 608. Furthermore, the classifier function C 610 is applied to the real output data y 604 for generating a first classification result C(y) 614. Additionally, the classifier function C 610 is applied to the synthetic output data G(x) 608 for generating a second classification result C(G(x)) 612.

Adapting the parameters of the generative function G 606 and the classifier function C 610 is based on minimizing a cost function by using the backpropagation algorithm, respectively. In this embodiment, the cost function K_{C} for the classifier function C 610 is K_{C} ∝-BCE (C(y), 1)-BCE (C(G(x), 0), wherein BCE denotes the binary cross entropy defined as BCE (z, z′)=z′·log (z)+(1−z′)·log (1−z). By using this cost function, both wrongly classifying real output data as synthetic (indicated by C(y)=0) and wrongly classifying synthetic output data as real (indicated as C(G(x)) 612≈1) increases the cost function K_{C} to be minimized. Furthermore, the cost function KG for the generator function G 606 is K_{G} ∝-BCE (C(G(x), 1)=−log (C(G(x). By using this cost function, correctly classified synthetic output data (indicated as C(G(x)) 612=0) leads to an increase of the cost function K_{G} to be minimized.

In particular, a recurrent machine learning model is a machine learning model whose output does not only depend on the input value and the parameters of the machine learning model adapted by the training process, but also on a hidden state vector, wherein the hidden state vector is based on previous inputs used on for the recurrent machine learning model. In particular, the recurrent machine learning model can comprise additional storage states or additional structures that incorporate time delays or comprise feedback loops.

In particular, the underlying structure of a recurrent machine learning model can be a neural network, which can be denoted as recurrent neural network. Such a recurrent neural network can be described as an artificial neural network where connections between nodes form a directed graph along a temporal sequence. In particular, a recurrent neural network can be interpreted as directed acyclic graph. In particular, the recurrent neural network can be a finite impulse recurrent neural network or an infinite impulse recurrent neural network (wherein a finite impulse network can be unrolled and replaced with a strictly feedforward neural network, and an infinite impulse network cannot be unrolled and replaced with a strictly feedforward neural network).

In particular, training a recurrent neural network can be based on the BPTT algorithm (acronym for “backpropagation through time”), on the RTRL algorithm (acronym for “real-time recurrent learning”) and/or on genetic algorithms.

By using a recurrent machine learning model input data comprising sequences of variable length can be used. In particular, this implies that the method cannot be used only for a fixed number of input datasets (and needs to be trained differently for every other number of input datasets used as input), but can be used for an arbitrary number of input datasets. This implies that the whole set of training data, independent of the number of input datasets contained in different sequences, can be used within the training, and that training data is not reduced to training data corresponding to a certain number of successive input datasets.

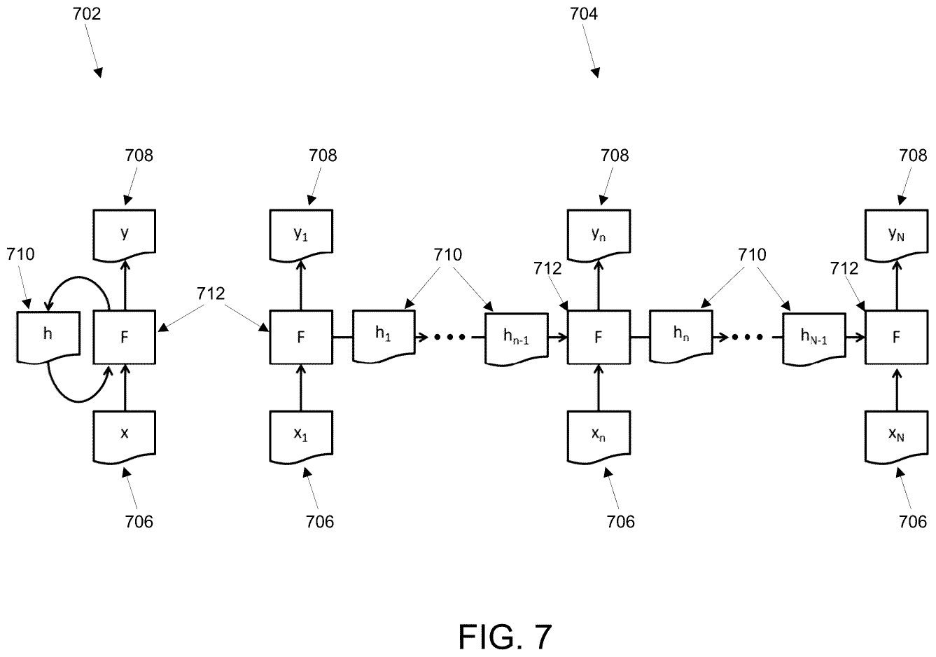

FIG. 7 shows the schematic structure of a recurrent machine learning model F, both in a recurrent representation 702 and in an unfolded representation 704, that may be used to implement one or more machine learning models described herein. The recurrent machine learning model takes as input several input datasets x, x₁, . . . , X_{N} 706 and creates a corresponding set of output datasets y, y₁, . . . , y_{N} 708. Furthermore, the output depends on a so-called hidden vector h, h₁, . . . , h_{N} 710, which implicitly comprises information about input datasets previously used as input for the recurrent machine learning model F 712. By using these hidden vectors h, h₁, . . . , h_{N} 710, a sequentiality of the input datasets can be leveraged.

In a single step of the processing, the recurrent machine learning model F 712 takes as input the hidden vector h_{N-1} created within the previous step and an input dataset xₙ. Within this step, the recurrent machine learning model F generates as output an updated hidden vector h_{N} and an output dataset yₙ. In other words, one step of processing calculates (yₙ, h_{N})=F(xₙ, h_{N-1}), or by splitting the recurrent machine learning model F 712 into a part F(y) calculating the output data and F^{(h)} calculating the hidden vector, one step of processing calculates yₙ=F^{(y)}(xₙ, h_{N-1}) and h_{N}=F^{(h)}(xₙ, h_{N-1}). For the first processing step, h₀ can be chosen randomly or filled with all entries being zero. The parameters of the recurrent machine learning model F 712 that were trained based on training datasets before do not change between the different processing steps.

In particular, the output data and the hidden vector of a processing step depend on all the previous input datasets used in the previous steps. yₙ=F^{(y)}(xₙ, F^{(h)}(xₙ₋₁, h_{N-2})) and h_{N}=F^{(h)}(xₙ, F^{(h)}(xₙ₋₁, h_{N-2})).

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIGS. 1-3. Certain steps or functions of the methods and workflows described herein, including one or more of the steps or functions of FIGS. 1-3, may be performed by a server or by another processor in a network-based cloud-computing system. Certain steps or functions of the methods and workflows described herein, including one or more of the steps of FIGS. 1-3, may be performed by a client computer in a network-based cloud computing system. The steps or functions of the methods and workflows described herein, including one or more of the steps of FIGS. 1-3, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIGS. 1-3, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 802 that may be used to implement systems, apparatuses, and methods described herein is depicted in FIG. 8. Computer 802 includes a processor 804 operatively coupled to a data storage device 812 and a memory 810. Processor 804 controls the overall operation of computer 802 by executing computer program instructions that define such operations. The computer program instructions may be stored in data storage device 812, or other computer readable medium, and loaded into memory 810 when execution of the computer program instructions is desired. Thus, the method and workflow steps or functions of FIGS. 1-3 can be defined by the computer program instructions stored in memory 810 and/or data storage device 812 and controlled by processor 804 executing the computer program instructions. For example, the computer program instructions can be implemented as computer executable code programmed by one skilled in the art to perform the method and workflow steps or functions of FIGS. 1-3. Accordingly, by executing the computer program instructions, the processor 804 executes the method and workflow steps or functions of FIGS. 1-3. Computer 802 may also include one or more network interfaces 806 for communicating with other devices via a network. Computer 802 may also include one or more input/output devices 808 that enable user interaction with computer 802 (e.g., display, keyboard, mouse, speakers, buttons, etc.).

Processor 804 may include both general and special purpose microprocessors, and may be the sole processor or one of multiple processors of computer 802. Processor 804 may include one or more central processing units (CPUs), for example. Processor 804, data storage device 812, and/or memory 810 may include, be supplemented by, or incorporated in, one or more application-specific integrated circuits (ASICs) and/or one or more field programmable gate arrays (FPGAs).

Data storage device 812 and memory 810 each include a tangible non-transitory computer readable storage medium. Data storage device 812, and memory 810, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 808 may include peripherals, such as a printer, scanner, display screen, etc. For example, input/output devices 808 may include a display device such as a cathode ray tube (CRT) or liquid crystal display (LCD) monitor for displaying information to the user, a keyboard, and a pointing device such as a mouse or a trackball by which the user can provide input to computer 802.

An image acquisition device 814 can be connected to the computer 802 to input image data (e.g., medical images) to the computer 802. It is possible to implement the image acquisition device 814 and the computer 802 as one device. It is also possible that the image acquisition device 814 and the computer 802 communicate wirelessly through a network. In a possible embodiment, the computer 802 can be located remotely with respect to the image acquisition device 814.

Any or all of the systems, apparatuses, and methods discussed herein may be implemented using one or more computers such as computer 802.

One skilled in the art will recognize that an implementation of an actual computer or computer system may have other structures and may contain other components as well, and that FIG. 8 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving one or more input medical images of an anatomical object of a patient acquired according to an imaging protocol; detecting one or more imaging errors in the one or more input medical images using one or more machine learning based networks; modifying the imaging protocol based on the one or more detected imaging errors; and outputting the modified imaging protocol.

Illustrative embodiment 2. The computer-implemented method of illustrative embodiment 1, wherein detecting one or more imaging errors in the one or more input medical images using one or more machine learning based networks comprises: detecting one or more imaging artifacts in the one or more input medical images using one or more machine learning based imaging artifact detection networks.

Illustrative embodiment 3. The computer-implemented method of illustrative embodiment 2, wherein the one or more imaging artifacts comprise at least one of image-based artifacts or k-space-based artifacts.

Illustrative embodiment 4. The computer-implemented method of any one of illustrative embodiments 2-3, wherein the one or more machine learning based imaging artifact detection networks are trained using synthetic images with imaging artifacts generated using a generator network, wherein the generator network receives as input real images and image artifact settings and generates as output the synthetic images with the imaging artifacts.

Illustrative embodiment 5. The computer-implemented method of any one of illustrative embodiments 2-4, wherein the one or more machine learning based imaging artifact detection networks are trained using synthetic images with imaging artifacts generated using a first generator network and a second generator network, wherein the first generator network receives as input real k-space data and generates as output synthetic k-space data, the synthetic k-space data being reconstructed into the synthetic images with the imaging artifacts, and wherein the second generator network receives as input real images and generates as output the synthetic images with the imaging artifacts.

Illustrative embodiment 6. The computer-implemented method of illustrative embodiment 5, wherein the first generator network and the second generator network are trained to enforce consistency between synthetic k-space training data generated by the first generator network and synthetic training images generated by the second generator network.

Illustrative embodiment 7. The computer-implemented method of any one of illustrative embodiments 1-6, wherein the one or more input medical images comprise one or more raw medical images and a plurality of post-processed medical images, and detecting one or more imaging errors in the one or more input medical images using one or more machine learning based networks comprises: detecting one or more post-processing errors in one or more of the plurality of post-processed medical images using a machine learning based post-processing error detection network, wherein the machine learning based post-processing error detection network receives as input the one or more raw medical images and the plurality of post-processed medical images and generates as output a plausibility classification for each of the plurality of post-processed medical images.

Illustrative embodiment 8. The computer-implemented method of any one of illustrative embodiments 1-7, wherein detecting one or more imaging errors in the one or more input medical images using one or more machine learning based networks comprises: detecting one or more imaging artifacts in the one or more input medical images; and propagating the one or more detected imaging artifacts to a post-processing error using a mapping function.

Illustrative embodiment 9. The computer-implemented method of claim 1, further comprising: automatically reacquiring or reprocessing the one or more input medical images according to the modified imaging protocol to acquire one or more additional medical images having the one or more detected imaging artifacts mitigated.

Illustrative embodiment 10. An apparatus comprising: means for receiving one or more input medical images of an anatomical object of a patient acquired according to an imaging protocol; means for detecting one or more imaging errors in the one or more input medical images using one or more machine learning based networks; means for modifying the imaging protocol based on the one or more detected imaging errors; and means for outputting the modified imaging protocol.

Illustrative embodiment 11. The apparatus of illustrative embodiment 10, wherein the means for detecting one or more imaging errors in the one or more input medical images using one or more machine learning based networks comprises: means for detecting one or more imaging artifacts in the one or more input medical images using one or more machine learning based imaging artifact detection networks.

Illustrative embodiment 12. The apparatus of illustrative embodiment 11, wherein the one or more imaging artifacts comprise at least one of image-based artifacts or k-space-based artifacts.

Illustrative embodiment 13. The apparatus of any one of illustrative embodiments 11-12, wherein the one or more machine learning based imaging artifact detection networks are trained using synthetic images with imaging artifacts generated using a generator network, wherein the generator network receives as input real images and image artifact settings and generates as output the synthetic images with the imaging artifacts.

Illustrative embodiment 14. The apparatus of any one of illustrative embodiments 11-13, wherein the one or more machine learning based imaging artifact detection networks are trained using synthetic images with imaging artifacts generated using a first generator network and a second generator network, wherein the first generator network receives as input real k-space data and generates as output synthetic k-space data, the synthetic k-space data being reconstructed into the synthetic images with the imaging artifacts, and wherein the second generator network receives as input real images and generates as output the synthetic images with the imaging artifacts.

Illustrative embodiment 15. The apparatus of illustrative embodiment 14, wherein the first generator network and the second generator network are trained to enforce consistency between synthetic k-space training data generated by the first generator network and synthetic training images generated by the second generator network.

Illustrative embodiment 16. A non-transitory computer-readable storage medium comprising instructions which, when executed by a computer, cause the computer to carry out operations comprising: receiving one or more input medical images of an anatomical object of a patient acquired according to an imaging protocol; detecting one or more imaging errors in the one or more input medical images using one or more machine learning based networks; modifying the imaging protocol based on the one or more detected imaging errors; and outputting the modified imaging protocol.

Illustrative embodiment 17. The non-transitory computer-readable storage of illustrative embodiment 16, wherein detecting one or more imaging errors in the one or more input medical images using one or more machine learning based networks comprises: detecting one or more imaging artifacts in the one or more input medical images using one or more machine learning based imaging artifact detection networks.

Illustrative embodiment 18. The non-transitory computer-readable storage of any one of illustrative embodiments 16-17, wherein the one or more input medical images comprise one or more raw medical images and a plurality of post-processed medical images, and detecting one or more imaging errors in the one or more input medical images using one or more machine learning based networks comprises: detecting one or more post-processing errors in one or more of the plurality of post-processed medical images using a machine learning based post-processing error detection network, wherein the machine learning based post-processing error detection network receives as input the one or more raw medical images and the plurality of post-processed medical images and generates as output a plausibility classification for each of the plurality of post-processed medical images.

Illustrative embodiment 19. The non-transitory computer-readable storage of any one of illustrative embodiments 16-18, wherein detecting one or more imaging errors in the one or more input medical images using one or more machine learning based networks comprises: detecting one or more imaging artifacts in the one or more input medical images; and propagating the one or more detected imaging artifacts to a post-processing error using a mapping function.

Illustrative embodiment 20. The non-transitory computer-readable storage of any one of illustrative embodiments 16-19, the operations further comprising: automatically reacquiring or reprocessing the one or more input medical images according to the modified imaging protocol to acquire one or more additional medical images having the one or more detected imaging artifacts mitigated.

1. A computer-implemented method comprising:
receiving one or more input medical images of an anatomical object of a patient acquired according to an imaging protocol;
detecting one or more imaging errors in the one or more input medical images using one or more machine learning based networks;
modifying the imaging protocol based on the one or more detected imaging errors; and
outputting the modified imaging protocol.

2. The computer-implemented method of claim 1, wherein detecting one or more imaging errors in the one or more input medical images using one or more machine learning based networks comprises:
detecting one or more imaging artifacts in the one or more input medical images using one or more machine learning based imaging artifact detection networks.

3. The computer-implemented method of claim 2, wherein the one or more imaging artifacts comprise at least one of image-based artifacts or k-space-based artifacts.

4. The computer-implemented method of claim 2, wherein the one or more machine learning based imaging artifact detection networks are trained using synthetic images with imaging artifacts generated using a generator network,
wherein the generator network receives as input real images and image artifact settings and generates as output the synthetic images with the imaging artifacts.

5. The computer-implemented method of claim 2, wherein the one or more machine learning based imaging artifact detection networks are trained using synthetic images with imaging artifacts generated using a first generator network and a second generator network,
wherein the first generator network receives as input real k-space data and generates as output synthetic k-space data, the synthetic k-space data being reconstructed into the synthetic images with the imaging artifacts, and
wherein the second generator network receives as input real images and generates as output the synthetic images with the imaging artifacts.

6. The computer-implemented method of claim 5, wherein the first generator network and the second generator network are trained to enforce consistency between synthetic k-space training data generated by the first generator network and synthetic training images generated by the second generator network.

7. The computer-implemented method of claim 1, wherein the one or more input medical images comprise one or more raw medical images and a plurality of post-processed medical images, and detecting one or more imaging errors in the one or more input medical images using one or more machine learning based networks comprises:
detecting one or more post-processing errors in one or more of the plurality of post-processed medical images using a machine learning based post-processing error detection network,
wherein the machine learning based post-processing error detection network receives as input the one or more raw medical images and the plurality of post-processed medical images and generates as output a plausibility classification for each of the plurality of post-processed medical images.

8. The computer-implemented method of claim 1, wherein detecting one or more imaging errors in the one or more input medical images using one or more machine learning based networks comprises:
detecting one or more imaging artifacts in the one or more input medical images; and
propagating the one or more detected imaging artifacts to a post-processing error using a mapping function.

9. The computer-implemented method of claim 1, further comprising:
automatically reacquiring or reprocessing the one or more input medical images according to the modified imaging protocol to acquire one or more additional medical images having the one or more detected imaging artifacts mitigated.

10. An apparatus comprising:
means for receiving one or more input medical images of an anatomical object of a patient acquired according to an imaging protocol;
means for detecting one or more imaging errors in the one or more input medical images using one or more machine learning based networks;
means for modifying the imaging protocol based on the one or more detected imaging errors; and
means for outputting the modified imaging protocol.

11. The apparatus of claim 10, wherein the means for detecting one or more imaging errors in the one or more input medical images using one or more machine learning based networks comprises:
means for detecting one or more imaging artifacts in the one or more input medical images using one or more machine learning based imaging artifact detection networks.

12. The apparatus of claim 11, wherein the one or more imaging artifacts comprise at least one of image-based artifacts or k-space-based artifacts.

13. The apparatus of claim 11, wherein the one or more machine learning based imaging artifact detection networks are trained using synthetic images with imaging artifacts generated using a generator network,
wherein the generator network receives as input real images and image artifact settings and generates as output the synthetic images with the imaging artifacts.

14. The apparatus of claim 11, wherein the one or more machine learning based imaging artifact detection networks are trained using synthetic images with imaging artifacts generated using a first generator network and a second generator network,
wherein the first generator network receives as input real k-space data and generates as output synthetic k-space data, the synthetic k-space data being reconstructed into the synthetic images with the imaging artifacts, and
wherein the second generator network receives as input real images and generates as output the synthetic images with the imaging artifacts.

15. The apparatus of claim 14, wherein the first generator network and the second generator network are trained to enforce consistency between synthetic k-space training data generated by the first generator network and synthetic training images generated by the second generator network.

16. A non-transitory computer-readable storage medium comprising instructions which, when executed by a computer, cause the computer to carry out operations comprising:
receiving one or more input medical images of an anatomical object of a patient acquired according to an imaging protocol;
detecting one or more imaging errors in the one or more input medical images using one or more machine learning based networks;
modifying the imaging protocol based on the one or more detected imaging errors; and
outputting the modified imaging protocol.

17. The non-transitory computer-readable storage of claim 16, wherein detecting one or more imaging errors in the one or more input medical images using one or more machine learning based networks comprises:
detecting one or more imaging artifacts in the one or more input medical images using one or more machine learning based imaging artifact detection networks.

18. The non-transitory computer-readable storage of claim 16, wherein the one or more input medical images comprise one or more raw medical images and a plurality of post-processed medical images, and detecting one or more imaging errors in the one or more input medical images using one or more machine learning based networks comprises:
detecting one or more post-processing errors in one or more of the plurality of post-processed medical images using a machine learning based post-processing error detection network,
wherein the machine learning based post-processing error detection network receives as input the one or more raw medical images and the plurality of post-processed medical images and generates as output a plausibility classification for each of the plurality of post-processed medical images.

19. The non-transitory computer-readable storage of claim 16, wherein detecting one or more imaging errors in the one or more input medical images using one or more machine learning based networks comprises:
detecting one or more imaging artifacts in the one or more input medical images; and
propagating the one or more detected imaging artifacts to a post-processing error using a mapping function.

20. The non-transitory computer-readable storage of claim 16, the operations further comprising:
automatically reacquiring or reprocessing the one or more input medical images according to the modified imaging protocol to acquire one or more additional medical images having the one or more detected imaging artifacts mitigated.
